(19) United States
(12) Patent Application Publication
(10) Pub. No.: US 2012/0197195 A1
(43) Pub. Date: Aug. 2, 2012

(54) Drug Delivery Device

(75) Inventors: Nils Basso, Frankfurt am Main (DE); Thomas Nagel, Tharandt (DE); Rene Richter, Tharandt (DE); Robert Witt, Dresden (DE)

(73) Assignee: Sanofi-Aventis Deutschland GmbH, Frankfurt am Main (DE)

(21) Appl. No.: 13/321,490

(22) PCT Filed: May 20, 2010
(86) PCT No.: PCT/EP2010/056976

§ 371 Date: Apr. 17, 2012

(30) Foreign Application Priority Data
May 20, 2009 (EP) 09006820.6

(51) Int. Cl. A61M 5/00

(52) U.S. Cl. 604/132

(57) ABSTRACT

A drug delivery device (1), comprising a flexible container (9) for holding a drug (18) and an electrically activatable deformable squeezing member (8). The flexible container (9) comprises a container outlet (17) for dispensing a dose of the drug (18) from the flexible container (9). The squeezing member (8) is activatable for dispensing the dose of the drug (18). When activated, the squeezing member (8) is arranged and configured to squeeze the flexible container (9), thereby expelling the dose of the drug (18) from the flexible container (9) through the container outlet (17).

This disclosure relates to a drug delivery device.

Usually, a drug delivery device is configured for setting and delivering one or more doses of a drug. Various mechanisms have previously been proposed to meet the specific requirements of a drug delivery device, e.g. with respect to dose accuracy.

Drug delivery devices are described in documents EP 1 177 802 B1 and US 2004/0138612 A1.

It is an object of the present disclosure to provide for an improved drug delivery device.

This object may be achieved by a drug delivery device according to the independent claim. Further features and advantageous embodiments are subject matter of the dependent claims.

According to one aspect a drug delivery device is provided comprising a flexible container for holding a drug. The drug delivery device comprises a squeezing member. The squeezing member may be deformable, preferably electrically activatable. Thus, the squeezing member may be an electrically activatable deformable squeezing member. The flexible container may comprise a container outlet for dispensing a dose of the drug from the flexible container. The squeezing member may be activatable for dispensing the dose of the drug. When activated, the squeezing member may be arranged and configured to squeeze the flexible container, in particular via its deformation, thereby expelling the dose of the drug from the flexible container through the container outlet.

The drug delivery device may be an injection device. The drug delivery device may be a pen-type device, e.g. a pen-type injector. The drug delivery device may comprise a housing. The drug delivery device may be configured to dispense fixed doses of a drug or variable, preferably user-settable doses of the drug.

According to an embodiment, the drug delivery device comprises an electrical power source for supplying electrical power to activate and preferably to control the deformation of the squeezing member.

The squeezing member may be an electrically activatable deformable member. Preferably, the squeezing member is reversibly deformable. When activated by means of the electrical power source, the squeezing member may deform, i.e. change its shape, e.g. the squeezing member contracts or expands, for squeezing the flexible container.

According to an embodiment, the squeezing member is a self-supporting member. When de-activated, e.g. when the power supply stops, the squeezing member may resume its original shape.

According to an embodiment, the squeezing member is a non-self-supporting member. The squeezing member may deform, in particular the squeezing member may change its outer shape, under the influence of its own weight, when the squeezing member is inactive, i.e. when the squeezing member is not activated to be deformed for dispensing the dose of the drug.

Due to the deformable squeezing member which squeezes the flexible container for expelling the drug from the flexible container a piston may be redundant. Hence, there is no friction between a piston and the container holding the drug. Accordingly, a drug delivery device of long lifetime is provided for which may have high dose accuracy.

According to an embodiment, the squeezing member encompasses the flexible container.

Preferably, the squeezing member delimits or defines an intermediate space. The flexible container may be arranged within said intermediate space. In particular, the flexible container may be surrounded by the squeezing member for effectively squeezing the flexible container once the squeezing member is activated. In this way, an optimal conversion of deformation, e.g. contraction or expansion, of the squeezing member into squeezing of the flexible container for expelling the drug from the flexible container may be achieved. Hence, a very efficient drug delivery device may be provided for.

According to an embodiment, the drug delivery device comprises a control member. The drug delivery device may comprise a device outlet. The control member may be provided between the container outlet and the device outlet. The control member may be configured to be operable to temporarily permit flow of the drug between the container outlet and the device outlet. The control member may comprise a valve.

The control member may be adapted to inhibit, to permit or to regulate the flow of the drug from the device outlet according to a dose of the drug which was set by a user. Preferably, the control member is configured to prevent flow of the drug from the flexible container, in particular from the container outlet, to the device outlet when the squeezing member is inactive, e.g. when no electrical power is supplied to activate the squeezing member.

According to an embodiment, the device comprises an electrical control unit. The control unit may be configured to control the activation of the squeezing member. Additionally or alternatively, the control unit may be configured to control the activation of the control member.

According to an embodiment, the electrically activatable deformable squeezing member is configured to adapt its shape to the outer shape of the flexible container. With the squeezing member being deformable, adapting its shape to the outer shape of the flexible container when it is activated and deformed, an optimal conversion of deformation of the squeezing member into compression of the flexible container may be achieved and hence, an effective and reliable drug delivery device may be provided for.

According to an embodiment, the drug delivery device comprises a counterpart. The supplied electrical power may cause the squeezing member to deform. Thereby, the squeezing member may push the flexible container against the counterpart for squeezing the flexible container.

According to an embodiment, the electrically activatable deformable squeezing member is an artificial muscle.

Preferably, the squeezing member is configured to act like a human muscle. The squeezing member may deform peristaltically for effectively squeezing the flexible container.

According to an embodiment, the electrically activatable deformable squeezing member comprises an electro-active polymer. An electro-active polymer may facilitate provision of a squeezing member acting like a human muscle.

According to an embodiment, the electrically activatable deformable squeezing member comprises a piezo-electric ceramic. By using a piezo-electric ceramic an especially cost-effective squeezing member and consequently, a cost-effective drug delivery device may be achieved.

According to an embodiment, the electrically activatable deformable squeezing member comprises a shape memory alloy. This may facilitate provision of a drug delivery device which may be easily adapted to the user's demands.

According to an embodiment, the flexible container is collapsible.

Preferably, the flexible container is deformed when force is exerted on it, e.g. when the drug is forced out of the flexible container. The flexible container may be configured to not return to its original shape after the dose has been delivered. This may facilitate delivering the drug even when the flexible container was almost emptied.

According to an embodiment, the drug delivery device comprises a rigid element. The rigid element may be arranged within the squeezing member. The rigid element may be rigid such as compared to the flexible container.

The rigid element may diminish the amount of non-dispensable drug remaining in the flexible container after the last dose was dispensed. When activated, the deformed squeezing member may press a wall of the flexible container and a wall of the rigid element against one another.

Of course, the subject matter of the different embodiments and aspects described above may be combined with each other.

Further features and refinements become apparent from the following description of the exemplary embodiments in connection with the accompanying figures.

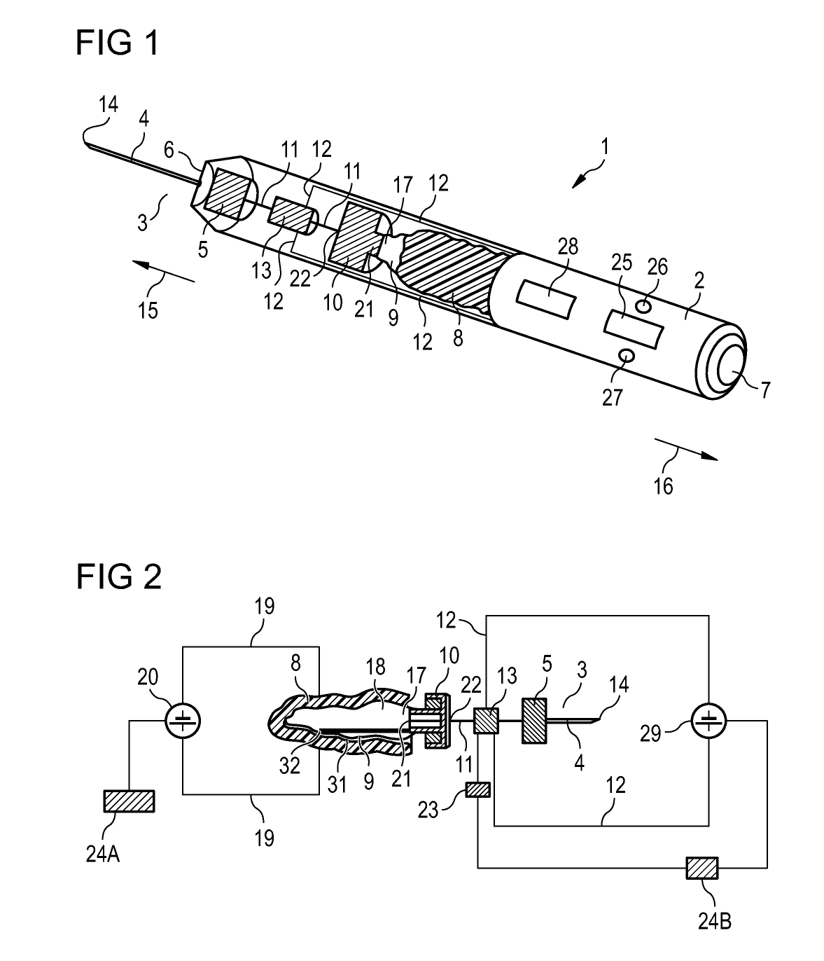

FIG. 1 schematically shows a sectional side view of an exemplary embodiment of a drug delivery device,

FIG. 2 schematically shows a sectional side view of a part of the drug delivery device of FIG. 1, and

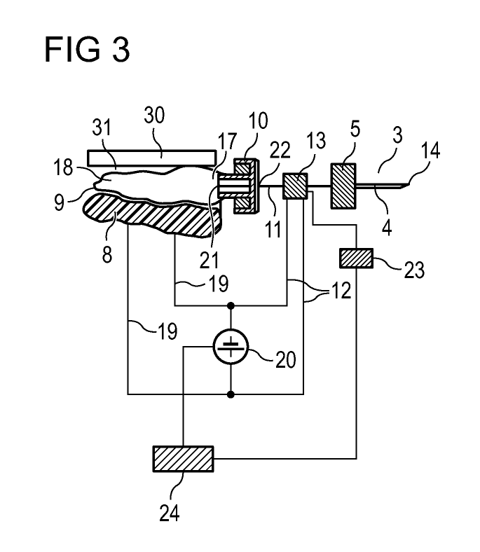

FIG. 3 schematically shows a sectional side view of a part of a drug delivery device according to another embodiment.

Like elements, elements of the same kind and identically acting elements may be provided with the same reference numerals in the figures.

In FIG. 1 a drug delivery device 1 is shown. The drug delivery device 1 comprises a housing 2. The drug delivery device 1 has a needle assembly 3 comprising a needle 4 and preferably a needle holder 5. The needle 4 may be attached to the needle holder 5. The needle holder 5 is attached to the housing 2. The drug delivery device 1 comprises a device outlet 6.

The device 1 comprises a squeezing member 8. Preferably, the squeezing member is a deformable member. The deformation of the squeezing member may be electrically activatable. The device 1 comprises a flexible container 9. The squeezing member 8 may define an intermediate space (see intermediate space 31 in FIGS. 2 and 3, for example). The flexible container 9 may be arranged in the intermediate space. Preferably, the squeezing member 8 encompasses the flexible container 9. The flexible container 9 may hold a plurality of doses of a drug (see drug 18 in FIGS. 2 and 3). Preferably, the drug is a liquid medication, comprising, for example, insulin, like short-acting or long acting-insulin, heparin or growth hormones.

The drug delivery device 1 has a dispensing end 14. The drug delivery device 1 comprises a conductor 12. The drug delivery device 1 comprises a control member 13. The control member 13 may be a valve, for example. The control member 13 may be provided between the flexible container 9 and the dispensing end 14 of the drug delivery device 1. The conductor 12 may be electrically conductively connected to the control member 13. The drug delivery device 1 comprises a connector 10. The connector 10 has a first end 21 and a second end 22. The drug delivery device 1 comprises a tube 11. The drug may be dispensed from the flexible container 9 and flow via the tube 11 towards and through the device outlet 6. A first part of the tube 11 may extend between the flexible container 9 and the control member 13. A second part of the tube 11 may further extend between the control member 13 and the needle assembly 3 of the drug delivery device 1.

The drug delivery device 1 comprises a display 25. The display 25 may be configured to display the size of a dose set by a user, for example. The drug delivery device 1 comprises at least one dose button for setting a dose of the drug. In this embodiment, the drug delivery device 1 has a first dose button 26 and a second dose button 27. In this embodiment, the drug delivery device 1 comprises a flow rate display 28. The drug delivery device 1 has at least one activation button 7.

The drug delivery device 1 may be a pen-type device, in particular a pen-type injector. The device 1 may be a disposable or a reusable device and may be configured to dispense fixed doses of the drug or variable, preferably user-settable, doses of the drug. The drug delivery device 1 may comprise the needle 4. Alternatively, the drug delivery device 1 may be a needle-free device (not shown in FIG. 1).

The housing 2 may be designed to enable a safe and comfortable handling of the drug delivery device 1. The housing 2 may be designed to house, fix, protect or guide inner components of the drug delivery device 1, e.g. flexible container 9, squeezing member 8, needle assembly 3. Preferably, the housing 2 limits or prevents the exposure of the inner components and of the drug to contaminants such as liquid, dirt or dust. The housing 2 may be a unitary or a multipart component. The housing 2 may comprise a tubular or cylindrical shape, as shown in FIG. 1. Alternatively, the housing 2 may comprise a non-tubular shape.

The flexible container 9 may hold a plurality of doses of the drug. Preferably, the flexible container 9 is collapsible. The flexible container 9 is deformed when force is exerted on it by the squeezing member 8. The drug may be expelled from the flexible container 9 when the flexible container 9 is deformed (see also the description of setting and delivering a dose of the drug as described in connection with FIG. 2). The flexible container 9 may be configured to not return to an original shape which the flexible container 9 had before force was exerted on it. This may facilitate delivering the drug when the flexible container 9 is almost emptied, e.g. after having delivered already a plurality of doses of the drug from the flexible container 9. The flexible container 9 may be a flexible bag which may comprise or be made of a flexible, preferably non-elastic, plastic material, for example. The flexible container 9 may be lighter than an ordinary container for holding the drug, thus enabling provision of an easily transportable drug delivery device 1.

The flexible container 9 may comprise a container outlet 17. The container outlet 17 may be, permanently or releasably, fixed to the connector 10, enabling flow of the drug from the flexible container 9 via the container outlet 17 towards and through the connector 10. The connector 10 and the flexible container 9 may form a container unit. The container unit may be more robust than the flexible container 9 taken alone. The connector 10 may serve for securing the container unit within the drug delivery device 1.

The connector 10 comprises the first end 21 and the second end 22. In this embodiment, the first end 21 corresponds to the inlet and the second end 22 corresponds to the outlet of the connector 10. The first end 21 of the connector 10 may be connected with the container outlet 17 of the flexible container 9 and the second end 22 may be connected with tube 11, in particular with the first part of the tube 11.

The connector 10 may be fixed to the housing 2, e.g. by means of snapping elements (not explicitly shown in FIG. 1). The snapping elements may prevent axial movement of the connector 10 with respect to the housing 2. Accordingly, the snapping elements may prevent movement of the container unit, i.e. of the connector 10 and the flexible container 9, with respect to the housing 2. If all of the doses of the drug that once were in the flexible container 9 have been dispensed, the container unit may be unsecured from the housing 2, thereby allowing removal of the connector 10 together with the flexible container 9 from the drug delivery device 1 for introducing a replacement container and, if applicable, a replacement connector, into the drug delivery device 1.

The device 1 comprises the squeezing member 8. The squeezing member 8 may be deformable. Preferably, the deformation of the squeezing member 8 is electrically activatable. The squeezing member 8 may be deformable and activatable by means of an electrical power source (see FIGS. 2 and 3). Upon activation the squeezing member 8 may be configured to be deformed, i.e. to change its shape, e.g. to contract or to expand.

The deformable squeezing member 8 may be an artificial muscle acting, i.e. deforming, similar to a human muscle. The squeezing member 8 may deform peristaltically for squeezing the flexible container 9. The squeezing member 8 may be a compact and space-saving member, enabling provision of a small drug delivery device.

The squeezing member 8 may be self-supporting, for example. If self-supporting, the squeezing member 8 may not change its outer shape under the influence of its own weight when the squeezing member 8 is inactive. This may have the advantage, that the squeezing member 8 is prevented from collapsing when inactive and hence, it may be prevented that the squeezing member 8 exerts pressure on the flexible container 9 due to its own weight when the squeezing member 8 is not deformed for dispensing a dose of the drug. Hence, control member 13 may be redundant as the stability of the squeezing member 8 on its own may already prevent unintentional flow of the drug from the flexible container 9 to the dispensing end 14 of the device 1. In particular, the stable squeezing member 8 may prevent flow of the drug when the squeezing member 8 is inactive. Alternatively, the squeezing member 8 may be non-self-supporting. Thus, the squeezing member 8 may change its outer shape under the influence of its own weight, in particular when the squeezing member 8 is inactive.

The flexible container 9 may be surrounded, in particular encompassed, by the squeezing member 8. The flexible container 9 may be arranged in the intermediate space (see intermediate space 31 in FIGS. 2 and 3) defined by the squeezing member 8. The squeezing member 8 may adapt its shape to the outer shape of the flexible container 9 once the squeezing member 8 is activated. If the squeezing member 8 is a non-self-supporting member, the squeezing member 8 may adapt its shape to the outer shape of the flexible container 9 in the inactive state, as well. The squeezing member 8 may be a re-usable member.

The squeezing member 8 is configured to deform when delivering the dose of the drug. Thereby, the squeezing member reduces the intermediate space in which the flexible container is arranged 9, thus exerting pressure onto the flexible container 9. Thereby, the flexible container 9 is squeezed and the dose of the drug is expelled from the flexible container 9 through the container outlet 17.

The drug delivery device 1 may comprise an electrical power source (see power source 20, FIG. 2) for supplying power to activate the squeezing member 8. The squeezing member 8 may comprise an electrically activatable deformable material, e.g. a material contracting or expanding when electrical power is supplied to it. The material may be suitable to transform supplied electrical energy into mechanical work, for peristaltically forcing the drug out of the flexible container 9. Said material may comprise an electro-active polymer (EAP). Alternatively or additionally, the material may comprise an electric ceramic (EAC), for example a lead-zirconate-titanate(Pb(Zr,Ti)O₃) or barium-titanate (BaTiO₃) crystal structure. Alternatively or additionally, said material may comprise a piezo-electric ceramic.

By using a piezo-electric ceramic, an especially cost-effective squeezing member 8 and consequently, a cost-effective drug delivery device 1 may be achieved.

An EAP squeezing member 8 may have the advantage of being particularly elastic, thus permitting a free shaping of the EAP squeezing member 8.

An EAC squeezing member 8 may have the advantage to facilitate provision of a self-supporting squeezing member 8, for example. Accordingly, a squeezing member 8 comprising an electric ceramic may keep its outer shape under the influence of its own weight even after the electrical power supply has ceased, i.e. when the squeezing member 8 was de-activated. Consequently, squeezing of the flexible container 9 and hence, delivering of a dose of the drug may be prevented even without provision of control member 13 when the squeezing member 8 is inactive.

Alternatively or additionally, the squeezing member 8 may comprise a shape memory alloy (SMA), for example a nickel-titanate (NiTi) crystal structure, or a copper-aluminium, or a copper-aluminium-nickel, or a iron-nickel-aluminium, or a SMA-polymer. A squeezing member 8 comprising an SMA may provide for a particularly flexible “all-purpose” drug delivery device 1, e.g. a device usable for dispensing very small doses, for example doses of 1 IU or less, as well as for dispensing very large doses, such as doses of 50 IU or greater, or any dose in-between, thereby providing high dose accuracy.

When removing the power from the squeezing member 8, i.e. when de-activating the squeezing member 8, the squeezing member 8 may be configured to return to an inactive state, thereby enlarging the intermediate space the flexible container 9 is arranged in and hence, reducing or removing the pressure exerted on the flexible container 9. In particular, the pressure exerted on the flexible container 9 when the squeezing member 8 is inactive is smaller than the pressure exerted on the flexible container 9 when the squeezing member 8 is active. Preferably, the squeezing member 8 is configured to expand or contract to the inactive state after the set dose has been delivered completely.

For supplying electrical power to activate the squeezing member 8 the drug delivery device 1 may comprise a conductor (see conductor 19 in FIG. 2) to electrically conductively connect the squeezing member 8 to the electrical power source. The electrical power source (not explicitly shown in FIG. 1) may comprise at least one accumulator or at least one battery, for example a coin-type battery.

The drug delivery device 1 comprises the control member 13. The control member 13 may be activated, e.g. opened, upon activation of the squeezing member 8. The control member 13 may be configured to control the amount of drug dispensed from the flexible container 9. The control member 13 may be provided between the container outlet 17 and the device outlet 6. The control member 13 may be configured to be operated to temporarily permit flow of the drug between the container outlet 17 and the device outlet 6. Power may be supplied to operate the control member 13 by means of the electrical power source 20 via conductor 12, for example. Power may be removed from the control member 13, e.g. the control member 13 may be closed, when the dose of the drug was delivered.

The drug delivery device 1 may comprise a control unit (see control unit 24A, 24B in FIG. 2 and control unit 24 in FIG. 3). The control unit may control the activation of the squeezing member 8. The control unit may control the activation of the control member 13. In particular, the control unit may be configured to control the state of the control member 13, e.g. whether the control member 13 is closed or opened. The control unit may comprise a logic controller chip, for example.

The drug delivery device 1 comprises the first dose button 26 and the second dose button 27 for setting a dose of the drug. The first dose button 26 may be configured to increase the size of the dose and the second dose button 27 may be configured to decrease the size of the dose. The currently set dose may be displayed in display 25.

The drug delivery device 1 comprises the activation button 7. The user may push onto the activation button 7 for initiating delivery of the set dose. Before pushing on the activation button 7 the squeezing member 8 may be inactive and the control member 13 may be closed. Operation of the activation button 7 may activate the control unit. Operation of the activation button 7 may initiate a dose dispensing process. Due to this embodiment, an easily manageable and user-friendly drug delivery device 1 may be achieved.

The control unit may actuate the electrical power source to supply power to activate the squeezing member 8. The control unit may actuate the electrical power source to supply power to activate the control member 13 for opening the control member 13 such that the drug can flow via the control member 13 to the dispensing end 14 of the device 1.

When activated the squeezing member 8 deforms and squeezes the flexible container 9. Thereby, the set dose of the drug is forced out of the flexible container 9. The drug flows towards and through the control member 13. The amount of the drug flowing through the control member 13 expediently corresponds to the dose previously set by the user.

To guarantee good dose accuracy a flow sensor (see flow sensor 23 in FIG. 2) may be provided for. The control member 13 may be connected to the flow sensor, for example. The flow sensor may feed back time-resolved information to the control unit. In particular, the flow sensor may feed-back information concerning the amount of the drug flowing through the control member 13 within a given period of time. The time-resolved amounts of the drug flowing through the control member 13 are summed up until the summed-up, i.e. the total, amount of the drug corresponds to the size of the dose which was set by the user. The drug delivery device 1 may comprise the flow rate display 28, which may display how much of the drug flows through the control member 13 within a given period of time. Alternatively, the amount of the drug flowing through the control member 13 may be displayed by display 25, for example. In this case the flow rate display 28 may be redundant.

When the set dose has been delivered completely the control member 13 may be closed. Preferably, the control unit actuates the electrical power source to remove power from the control member 13 after the set dose has been delivered completely for closing the control member 13. Hence, further flow of the drug via the tube 11 to the dispensing end 14 of the device 1 may be prevented.

Additionally or alternatively, the squeezing member 8 may be de-activated when the set dose has been delivered completely. Preferably, when the set dose has been delivered completely, the control unit may prompt the electrical power source to remove power from the squeezing member 8, causing the squeezing member 8 to deform to the inactive state. Hence, the drug no longer flows from the flexible container 9 via the tube 11 to the dispensing end 14 of the device 1.

According to another embodiment, the control member 13 may be controlled mechanically, for example via a valve-locking-screw (not explicitly shown in FIG. 1). A user may rotate the valve-locking-screw with respect to the housing 2 for opening, closing or regulating the control member 13, hence permitting, inhibiting or regulating the flow of the drug from the flexible container 9 via the control member 13 to the dispensing end 14 of the device 1.

FIG. 2 shows a sectional side view of a part of the drug delivery device of FIG. 1. In particular, FIG. 2 shows the flexible container 9 holding the drug 18. Features and advantageous embodiments described in conjunction with the description of FIG. 1 may also apply for the device described in conjunction with FIG. 2.

The drug delivery device 1 comprises the deformable squeezing member 8 which encompasses the flexible container 9. A squeezing member control unit 24A is provided for. The device 1 has a conductor 19 and an electrical power source 20. The electrical power source 20 may be configured to supply power to activate the squeezing member 8.

The drug delivery device 1 comprises a control member control unit 24B. The device 1 comprises flow sensor 23. The drug delivery device 1 comprises a power source 29. The power source 29 may be adapted to supply power to activate the control member 13.

The drug delivery device 1 comprises a rigid element 32 which may facilitate forcing even small doses of the drug out of the flexible container 9. The rigid element 32 may comprise a thin plate made of a rigid plastic material, for example. The rigid element 32 may be arranged in the intermediate space 31 abutting the flexible container 9 for facilitating squeezing of the almost emptied flexible container 9. Alternatively, as shown in FIG. 2, the rigid element 32 may be arranged within the flexible container 9. The rigid element 32 may be rigid such as compared to the flexible container 9. When activated, the deformed squeezing member 8 may press a wall of the flexible container 9 and the rigid element 32 against one another. Thereby, the rigid element 32 may diminish the amount of the non-dispensable drug remaining in the flexible container 9 after the last dose has been delivered.

According to the embodiment of the drug delivery device 1 described in connection with the description of FIG. 1, the flexible container 9 holding the drug 18 is arranged in the intermediate space 31 delimited by the squeezing member 8.

Control units 24A and 24B may comprise a respective control chip. The squeezing member control unit 24A is electrically conductively connected to the electrical power source 20, which is configured to supply power to activate the squeezing member 8. The control member control unit 24B is electrically conductively connected to the electrical power source 29, which is configured to supply power to activate the control member 13. The control member 13 is electrically conductively connected to the flow sensor 23, which is electrically conductively connected to the control member control unit 24.

In the following, operation for setting and delivering a dose of the drug 18 from the flexible container 9 is described.

For setting a dose the user pushes onto the first dose button 26 until the desired size of the dose of the drug 18 has been reached. The set dose is displayed in display 25 as described previously.

After having set the dose the user may push onto the at least one activation button 7 actuating the control units 24A, 24B to initiate delivery of the set dose of the drug 18. Preferably, the control member control unit 24B may be actuated via pushing onto a control member activation button (not explicitly shown in FIG. 2). Preferably, the squeezing member control unit 24A may be actuated via pushing onto a squeezing member activation button (not explicitly shown in FIG. 2).

The control member control unit 24B may activate operation of the control member 13. Alternatively, the user may open control member 13 mechanically or electrically via a separate circuit before pushing or after having pushed the squeezing member activation button, for example by means of the valve-locking-screw as described above.

The squeezing member control unit 24A may actuate the electrical power source 20 to supply power to activate the squeezing member 8. Due to the supplied power, the squeezing member 8 deforms. Therefore, the intermediate space 31 in which the flexible container 9 is arranged is reduced and the squeezing member 8 squeezes the flexible container 9, hence forcing the set dose of the drug 18 from the flexible container 9 through the container outlet 17 and through the connector 10 via the tube 11, the control member 13 and the device outlet 6 to the dispensing end 14 of the device 1.

Flow sensor 23 will hence measure how much of the drug 18 flows through the control member 13 within a given period of time. The measured amounts of the drug 18 may be displayed by the flow rate display 28, for example.

After the dose has been delivered completely the control member control unit 24B may actuate the electrical power source 29 to remove power from the control member 13 for closing the control member 13. Alternatively, the user may rotate the valve-locking-screw with respect to the housing 2 to close the control member 13. For this purpose, the control member control unit 24B may prompt the user to close the control member 13 manually by means of the valve-locking-screw for example via displaying the term “close valve” in the flow rate display 28.

In addition, the squeezing member control unit 24A may actuate the electrical power source 20 to remove power from the squeezing member 8. After having removed the power from the squeezing member 8, the squeezing member 8 may return to the inactive state or stop to contract or expand. Hence, the pressure may be removed from the flexible container 9.

As the control member 13 has been closed and the squeezing member 8 has returned to the inactive position, flow of the drug 18 from the dispensing end 14 no longer takes place until the user sets another dose of the drug.

FIG. 3 schematically shows a sectional side view of a part of a drug delivery device according to another embodiment. Features and advantageous embodiments described in conjunction with the description of FIGS. 1 and 2 may also apply for the device described in conjunction with FIG. 3. Operation of the squeezing member 8 may occur in the same way as described in connection with FIG. 2.

In this embodiment, the flexible container 9 may be arranged in an intermediate space 31 between the deformable squeezing member 8 and a counterpart 30. Supplied electrical power may cause the squeezing member 8 to deform. Thereby, the squeezing member 8 may diminish the intermediate space 31 and may push the flexible container 9 against the counterpart 30. Hence, the flexible container 9 is squeezed and the drug 18 is forced out of the flexible container 9.

As the flexible container 9 is arranged between the squeezing member 8 and the counterpart 30, the flexible container 9, in particular the container unit, may easily be removed from the drug delivery device 1 for replacing the flexible container 9 with a replacement container, for example, if all of the drug 18 once held in the flexible container 9 has been dispensed. For replacing the flexible container 9 temporary removal or even replacement of the squeezing member 8 may be redundant. This arrangement of the flexible container 9 and the squeezing member 8 may be especially suitable for being integrated in a re-usable drug delivery device 1.

The counterpart 30 may be any rigid, preferably non-activatable, member being suitable to act as counterpart. Preferably, the counterpart 30 comprises a thin, rigid plate, preferably made of plastic. The counterpart 30 may be secured against translatory movement with respect to the housing 2 of the drug delivery device 1. Preferably, the counterpart 30 is glued or screwed to the housing 2.

Both the squeezing member 8 and the control member 13 are electrically conductively connected to electrical power source 20. Hence, in contrast to the embodiment shown in FIG. 2, there is only one electrical power source for supplying power to activate the squeezing member 8 and the control member 13.

The electrical power source 20 is electrically conductively connected to the control unit 24. The control unit 24 is electrically conductively connected to the flow sensor 23, which is electrically conductively connected to the control member 13. In this embodiment, there is only one control unit, which is control unit 24, for operating the flow sensor 23, the power source 20 and hence, the squeezing member 8 and the control member 13.

With the drug delivery device 1 described herein above a good dose accuracy may be achieved. Thereby, the drug delivery device 1 may provide for small doses, for example doses of 5 IU or less, for example 1 IU, as well as for large doses, such as doses of 30 IU or greater, for example 50 IU.

Other implementations are within the scope of the following claims. Elements of different implementations may be combined to form implementations not specifically described herein.

The term “drug” or “medicament”, as used herein, means a pharmaceutical formulation containing at least one pharmaceutically active compound, wherein in one embodiment the pharmaceutically active compound has a molecular

weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

### REFERENCE NUMERALS

1 Drug delivery device
2 Housing
3 Needle assembly
4 Needle
5 Needle holder
6 Device outlet
7 Activation button
8 Squeezing member
9 Flexible container
10 Connector
11 Tube
12 Conductor
13 Control member
14 Dispensing end
15 Distal end
16 Proximal end
17 Container outlet
18 Drug
19 Conductor
20 Electrical power source
21 First end of connector
22 Second end of connector
23 Flow sensor
24 Control unit
24A Squeezing member control unit
24B Control member control unit
25 Display
26 First dose button
27 Second dose button
28 Flow rate display
29 Electrical power source
30 Counterpart
31 Intermediate space
32 Rigid element

1. A drug delivery device (1), comprising:
a flexible container (9) for holding a drug (18), wherein the flexible container (9) comprises a container outlet (17) for dispensing a dose of the drug (18) from the flexible container (9),
an electrically activatable deformable squeezing member (8), the squeezing member (8) being activatable for dispensing the dose of the drug (18), wherein,
when activated, the squeezing member (8) is arranged and configured to squeeze the flexible container (9), thereby expelling the dose of the drug (18) from the flexible container (9) through the container outlet (17).

2. The drug delivery device (1) according to claim 1,
wherein the drug delivery device (1) comprises an electrical power source (20) for supplying electrical power to activate deformation of the squeezing member (8).

3. The drug delivery device (1) according to any of the previous claims,
wherein the electrically activatable deformable squeezing member (8) encompasses the flexible container (9).

4. The drug delivery device (1) according to any of the previous claims,
which comprises a control member (13) and a device outlet (6), the control member (13) being provided between the container outlet (17) and the device outlet (6),
wherein the control member (13) is configured to be operable to temporarily permit flow of the drug (18) between the container outlet (17) and the device outlet (6).

5. The drug delivery device (1) according to claim 4,
wherein the control member (13) comprises a valve.

6. The drug delivery device (1) according to claim 4 and claim 5 or any claim depending thereof,
which comprises an electrical control unit (24), the control unit (24) being configured to control the activation of the squeezing member (8) and the activation of the control member (13).

7. The drug delivery device (1) according to any of the previous claims,
wherein the electrically activatable deformable squeezing member (8) is configured to adapt its shape to the outer shape of the flexible container (9).

8. The drug delivery device (1) according to any of the previous claims,
which comprises a counterpart (30), wherein the supplied electrical power causes the squeezing member (8) to deform, thereby pushing the flexible container (9) against the counterpart (30) for squeezing the flexible container (9).

9. The drug delivery device (1) according to any of the previous claims,
wherein the electrically activatable deformable squeezing member (8) is an artificial muscle.

10. The drug delivery device (1) according to any of the previous claims,
wherein the electrically activatable deformable squeezing member (8) comprises an electro-active polymer.

11. The drug delivery device according to any of claims 1 to 10,
wherein the electrically activatable deformable squeezing member (8) comprises a piezo-electric ceramic.

12. The drug delivery device according to any of claims 1 to 11,
wherein the electrically activatable deformable squeezing member (8) comprises a shape memory alloy.

13. The drug delivery device (1) according to any of the previous claims, wherein the flexible container (9) is collapsible.

14. The drug delivery device (1) according to any of the previous claims,
wherein the drug delivery device (1) comprises a rigid element (32) arranged within the squeezing member (8), wherein the rigid element (32) is rigid such as compared to the flexible container (9).

15. The drug delivery device (1) according to any of the previous claims,
wherein the drug delivery device (1) is a pen-type device.
